SEQUENCE LISTING (1 sequence)

SEQ ID NO: 1 - RNA, 22 nt, Homo sapiens
uagcuuaucagacugauguuga

(19) United States
(12) Patent Application Publication
(10) Pub. No.: US 2011/0033855 A1
(43) Pub. Date: Feb. 10, 2011

(54) METHOD FOR QUANTIFYING TARGET NUCLEIC ACID MOLECULES AND KIT FOR QUANTIFYING TARGET NUCLEIC ACID MOLECULES

(75) Inventors: Kunio HORI, Tokyo (JP); Hidetaka NAKATA, Tokyo (JP); Sayoko KOBAYASHI, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/851,262

(22) Filed: Aug. 5, 2010

(30) Foreign Application Priority Data
Aug. 7, 2009 (JP) 2009-184523

(51) Int. Cl. C12Q 1/68; G01N 33/566

(52) U.S. Cl. 435/6; 436/501

(57) ABSTRACT

The present invention is to provide a method for highly sensitively and precisely quantifying nucleic acid molecules in a sample. The method for quantifying target nucleic acid molecules in a nucleic acid-containing sample comprises: (a) preparing a sample solution comprising a nucleic acid-containing sample, a first nucleic acid molecule probe comprising a sequence complementary to the target nucleic acid molecule and conjugated with a first marker, and a second nucleic acid molecule probe comprising a sequence complementary to the first nucleic acid molecule probe and conjugated with a second marker; (b) denaturing nucleic acid molecules in this sample solution; (c) hybridizing them; (d) forming a covalent bond between two nucleic acid strands in the hybrid under a same condition, regarding the temperature and the salt concentration, as that of the hybrid formation; and (e) quantifying the target nucleic acid molecules by detecting a time course change in an optical characteristic of the first marker or the second marker in the sample solution, wherein an optical characteristic of at least either one of the first marker and the second marker is changed depending on whether or not the first nucleic acid molecule probe and the second nucleic acid molecule probe are hybridized.

### BACKGROUND OF THE INVENTION

1. Field of the Invention

The present invention relates to a method for quantifying target nucleic acid molecules in a nucleic acid-containing sample by utilizing fluorescence resonance energy transfer (FRET) and a photocrosslinking reaction.

2. Description of Related Art

Regarding highly sensitive methods for quantifying nucleic acid molecules in a sample, there are known methods which employ techniques for producing fluorescence signals by amplifying a target nucleic acid molecule through an enzymatic reaction with a polymerase, or by decomposing the thus yielded amplification product, that is, so-called quantitative PCR (polymerase chain reaction) techniques. There are some methods which employ the quantitative PCR techniques. Of these, widely conducted is a so-called TaqMan method in which a FRET-based probe is applied to the target nucleic acid molecule. For example, quantitative PCR using a FRET probe is being developed as a method for quantifying short RNA molecules of about 22-mer such as a micro RNA (for example, refer to Non-patent Document 1) and an siRNA for use in RNA interference (for example, refer to Non-patent Document 2).

As one of the nucleic acid detection methods using FRET probes, for example, there is disclosed a method in which a donor probe conjugated with a fluorescent substance serving as a donor of the fluorescence resonance energy and an acceptor probe conjugated with a quenching substance serving as an acceptor thereof are respectively hybridized to two adjacent regions in a target nucleic acid molecule, and then resultant FRET is detected to thereby detect the target nucleic acid molecule (for example, refer to Patent Documents 1 to 3). In addition, there is also disclosed a method in which a probe conjugated with both a fluorescent substance and a quenching substance is hybridized to a target nucleic acid molecule, after which the produced hybrid is decomposed by a double-stranded DNA specific-nuclease, and the resultant fluorescence is detected (for example, refer to Patent Document 4). There is also disclosed a so-called molecular torch method which uses a single-stranded nucleic acid having its 5′ end side and 3′ end side consisting of mutually complementary nucleotide sequences, and respectively labeled with a fluorescent substance and a quenching substance (for example, refer to Patent Document 5). In this method, when the single-stranded nucleic acid exists by itself, it falls into a quenched state because both ends are hybridized to form an intramolecular loop. However, when it is hybridized with a target nucleic acid molecule, the intramolecular loop is broken to emit fluorescence.

Besides, as a method for identifying and detecting nucleic acid molecules having mutually different nucleotide sequences, there is disclosed a method in which a double-stranded DNAβ serving as a reference and a double-stranded DNAβx serving as a detection target are prepared, a fluorescent substance as a first marker is given to the reference double-stranded DNA, and a second marker is designed so that energy transfer can occur between the first marker and the second marker to thereby yield fluorescence having another wavelength peak, by which a nucleotide difference between the DNAβx to be detected and the reference DNAβ can be detected (for example, refer to Patent Document 6).

Meanwhile, a large number of tools for more efficiently analyzing nucleic acid molecules are also under development. For example, a method has been developed in which a reactive functional group is introduced into a nucleotide serving as a component of an oligonucleotide, and a covalent bond is formed between the oligonucleotide and another oligonucleotide or molecule via the reactive functional group (crosslinking). For example, as a technique for covalently crosslinking nucleic acid molecules with use of a nucleotide derivative in which a reactive functional group has been introduced, there are disclosed methods which use 2-amino-6-vinylpurine (for example, refer to Non-patent Document 3), methods which use 3-cyanovinylcarbazole nucleoside that is a photoreactive nucleotide derivative (for example, refer to Non-patent Document 4 or 5), and the like.

### REFERENCES

### Patent Documents

Patent Document 1: Japanese Patent No. 4008996

Patent Document 2: PCT International Publication No. WO 98/13524 pamphlet

Patent Document 3: Japanese Patent No. 3188303

Patent Document 4: PCT International Publication No. WO 03/035864 pamphlet

Patent Document 5: Published Japanese Translation No. 2002-519073 of the PCT

### International Publication

Patent Document 6: Japanese Unexamined Patent Application, First Publication No. 2002-171974

### Non-Patent Documents

Non-patent Document 1: Alberts et al., Molecular Biology of the Cell, 5th edition, Garland Science, 2008, pp. 493 to 495
Non-patent Document 2: Chen et al., Nucleic Acids Research, 2005, Vol. 33, No. 20, p. e179
Non-patent Document 3: Shigeki SASAKI, YAKUGAKU ZASSHI, 2002, Vol. 122, No. 12, pp. 1081 to 1093
Non-patent Document 4: Fujimoto et al., Nucleic Acids Symposium Series, 2008, Vol. 52, pp. 423 to 424
Non-patent Document 5: Yoshimura et al., ORGANIC LETTERS, 2008, Vol. 10, No. 15, pp. 3227 to 3230

### SUMMARY OF THE INVENTION

In the prior art quantitative PCR methods, the characteristic of amplification with a polymerase varies depending on the type of the nucleotide sequence of the amplification target nucleic acid. Therefore, in order to quantitatively measure the concentration of target nucleic acid molecules, it is necessary to form a calibration curve per each type of the target nucleic acid molecules. This is problematic in that the number of steps has to be increased for the detection and thus the whole process becomes complicated. On the other hand, in the methods using FRET probes, the target nucleic acid molecule can be detected through detection of fluorescence emitted or quenched by FRET. However, the detected fluorescence may appear in the background at a certain fluorescence intensity because of an imperfect photoquenching action depending on the combination of fluorescent molecules. For this reason there is a problem in that the quantitativity becomes inferior particularly when the target nucleic acid molecules are at a low concentration.

Moreover, in detections utilizing hybridization, the produced hybrid is usually detected in a temperature condition for normal measurement (for example, room temperature or the like). Therefore, there is a problem in that nonspecific hybrids (hybrids formed by nonspecific hybridizations) can be easily formed during the operation for detecting the hybrid. However, it may be sometimes be difficult to discriminate between a nonspecific hybrid and a specific hybrid. For the discrimination between both parties, there is the difficulty that the hybridization condition has to be changed per each type of the target nucleic acid, and other similar difficulties.

It is an object of the present invention to provide a method with which nucleic acid molecules in a sample can be highly sensitively and precisely quantified.

The inventors of the present invention have conducted extensive studies in order to solve the aforementioned problems, and have found that in a target nucleic acid molecule quantification method with use of a FRET probe, direct quantitative analysis of target nucleic acid molecules in a sample can be achieved by performing the detection and analysis for individual molecules in the fluorescence intensity analysis of FRET energy transfer, and precise quantitative analysis of the target nucleic acid molecules in the sample can be achieved by hybridizing the FRET probe to the target nucleic acid molecule under a suitable condition for the specific hybridization, and subsequently forming a covalent bond between two nucleic acid strands in the thus formed hybrid without changing the temperature and the salt concentration of the reaction solution. This has led to the completion of the present invention.

That is, the present invention provides:

(1) a method for quantifying target nucleic acid molecules in a nucleic acid-containing sample, comprising:

(a) preparing a sample solution comprising a nucleic acid-containing sample, a first nucleic acid molecule probe conjugated with a first marker, and a second nucleic acid molecule probe conjugated with a second marker;

(b) denaturing nucleic acid molecules in the sample solution prepared in (a);

(c) hybridizing the nucleic acid molecules in said sample solution, after (b);

(d) forming a covalent bond between two nucleic acid strands in the thus formed hybrid under a same condition, regarding the temperature and the salt concentration of the sample solution, as that of the hybrid formation in (c), after (c); and

(e) quantifying said target nucleic acid molecules by detecting a time course change in an optical characteristic of the first marker or the second marker in said sample solution, after (d);

wherein said first nucleic acid molecule probe comprises a nucleotide sequence complementary to said target nucleic acid molecule, said second nucleic acid molecule probe comprises a nucleotide sequence complementary to said first nucleic acid molecule probe, and at least either one of said first marker and said second marker is a substance whose optical characteristic is changed depending on whether or not said first nucleic acid molecule probe and said second nucleic acid molecule probe are hybridized;
(2) the method for quantifying target nucleic acid molecules in a nucleic acid-containing sample target nucleic acid molecule quantification according to (1), wherein at least either one of said first marker and said second marker is a fluorescent dye, and said optical characteristic is detected through detection of a time course change in the fluorescence intensity emitting from said first marker or said second marker;
(3) the method for quantifying target nucleic acid molecules in a nucleic acid-containing sample target nucleic acid molecule quantification according to (1), wherein the reaction for forming the covalent bond in (d) is a photochemical reaction;
(4) the method for quantifying target nucleic acid molecules in a nucleic acid-containing sample target nucleic acid molecule quantification according to (3), wherein at least one nucleotide of said first nucleic acid molecule probe within the nucleotide sequence complementary to said target nucleic acid molecule is substituted by a photoreactive nucleotide derivative, and said covalent bond is fainted via said photoreactive nucleotide derivative;
(5) the method for quantifying target nucleic acid molecules in a nucleic acid-containing sample target nucleic acid molecule quantification according to (1), wherein at least one nucleotide of said first nucleic acid molecule probe in the nucleotide sequence complementary to said target nucleic acid molecule is substituted by 3-cyanovinylcarbazole nucleoside, and said covalent bond is formed by irradiation on said sample solution with light at 340 to 380 nm;
(6) the method for quantifying target nucleic acid molecules in a nucleic acid-containing sample target nucleic acid molecule quantification according to (1), wherein a temperature of said sample solution at the time of covalent bond formation in (d) is within a range of Tm value±3° C.;
(7) the method for quantifying target nucleic acid molecules in a nucleic acid-containing sample target nucleic acid molecule quantification according to (2), wherein said target nucleic acid molecules are quantified through detection of a change in the fluorescence intensity of molecules existing in the focal area of a confocal optical system, and subsequent statistical analysis thereof to calculate the number of molecules of the first nucleic acid molecule probe or the second nucleic acid molecule probe hybridizing to said target nucleic acid molecules;
(8) the method for quantifying target nucleic acid molecules in a nucleic acid-containing sample target nucleic acid molecule quantification according to (2), wherein said target nucleic acid molecules are quantified through detection of a fluctuation of the fluorescence intensity of molecules existing in the focal area in a confocal optical system, and subsequent statistical analysis thereof to calculate the number of molecules of the first nucleic acid molecule probe or the second nucleic acid molecule probe hybridizing to said target nucleic acid molecules;
(9) the method for quantifying target nucleic acid molecules in a nucleic acid-containing sample target nucleic acid molecule quantification according to (8), wherein the fluctuation of the fluorescence intensity is analyzed by autocorrelation analysis;
(10) the method for quantifying target nucleic acid molecules in a nucleic acid-containing sample target nucleic acid molecule quantification according to (1), wherein said first marker is a fluorescent substance and said second marker is a quenching substance for quenching fluorescence emitting from said fluorescent substance;
(11) the method for quantifying target nucleic acid molecules in a nucleic acid-containing sample target nucleic acid molecule quantification according to (1), wherein said second marker is a fluorescent substance and said first marker is a quenching substance for quenching fluorescence emitting from said fluorescent substance;
(12) the method for quantifying target nucleic acid molecules in a nucleic acid-containing sample target nucleic acid molecule quantification according to (1), wherein said first marker and said second marker are fluorescent dyes;
(13) the method for quantifying target nucleic acid molecules in a nucleic acid-containing sample target nucleic acid molecule quantification according to (1), wherein said target nucleic acid molecule is a micro RNA;
(14) the method for quantifying target nucleic acid molecules in a nucleic acid-containing sample target nucleic acid molecule quantification according to (1), wherein said target nucleic acid molecule is an siRNA; and
(15) a target nucleic acid molecule quantification kit comprising:

a first nucleic acid molecule probe comprising a nucleotide sequence complementary to the target nucleic acid molecule and conjugated with a first marker, and

a second nucleic acid molecule probe comprising a nucleotide sequence complementary to the first nucleic acid molecule probe and conjugated with a second marker;

wherein at least either one of said first marker and said second marker is a molecule whose optical characteristic is changed depending on whether or not said first nucleic acid molecule probe and said second nucleic acid molecule probe are hybridized, and at least one nucleotide of said first nucleic acid molecule probe within the nucleotide sequence complementary to said target nucleic acid molecule is substituted by a photoreactive nucleotide derivative.

The target nucleic acid molecule quantification method of the present invention utilizes FRET between a probe conjugated with a first marker and a probe conjugated with a second marker whose optical characteristic is changed as it comes closer to the first marker, and performs the fluorescence intensity analysis of FRET energy transfer for individual molecules in a sample. Therefore, the target nucleic acid molecules can be directly quantified.

In addition, in the target nucleic acid molecule quantification method of the present invention forms a covalent bond is formed between the target nucleic acid molecule and the first nucleic acid molecule probe on completion of specific hybridization therebetween, by which the formed hybrid can be stably maintained until the time of the fluorescence intensity analysis of energy transfer. Therefore, the formation of nonspecific hybrids can be effectively inhibited as well as improving the specificity and the preciseness in the detection and the quantification of the target nucleic acid molecules.

For this reason, with use of the target nucleic acid molecule quantification method of the present invention, the target nucleic acid molecules can be precisely quantified without considering; the characteristic of the nucleotide sequence of the target nucleic acid molecule, a condition such as a concentration in the nucleic acid-containing sample, a temperature condition at the time of the fluorescence intensity analysis of FRET energy transfer, and the like.

### BRIEF DESCRIPTION OF THE DRAWINGS

FIG. 1 is a schematic diagram showing the effect of covalent bond formation (crosslink) between a target nucleic acid molecule and a first nucleic acid molecule probe which constitute a hybrid.

FIG. 2 shows one aspect of the target nucleic acid molecule quantification method of the present invention in which a fluorescent dye is used as a first marker and a dark quencher is used as a second marker.

FIG. 3 shows another aspect of the target nucleic acid molecule quantification method of the present invention in which fluorescent dyes are used as a first marker and a second marker.

FIG. 4 shows results of FIDA analysis in Example 1.

FIG. 5 shows results of FIDA analysis in Example 2.

### DETAILED DESCRIPTION OF THE INVENTION

The method of the present invention is for quantifying target nucleic acid molecules in a nucleic acid-containing sample, wherein the detection and the analysis are performed for individual molecules in the fluorescence intensity analysis of FRET energy transfer. Specifically it is a method which uses a first nucleic acid molecule probe comprising a nucleotide sequence complementary to the target nucleic acid molecule, and a second nucleic acid molecule probe comprising a nucleotide sequence complementary to the first nucleic acid molecule probe, wherein the target nucleic acid molecules are quantified through precise detection of a hybrid between the target nucleic acid molecule and the first nucleic acid molecule probe by competitively hybridizing the target nucleic acid molecule to the first nucleic acid molecule probe, and the second nucleic acid molecule probe to the first nucleic acid molecule probe.

The hybrid between the target nucleic acid molecule and the first nucleic acid molecule probe is detected by utilizing FRET occurring between the first marker conjugated to the first nucleic acid molecule probe and the second marker conjugated to the second nucleic acid molecule probe. The second marker conjugated to the second nucleic acid molecule probe is set to be a substance whose optical characteristic is changed as it comes closer to the first marker conjugated to the first nucleic acid molecule probe. Specifically, as for the first marker and the second marker of the present invention, at least either one of the first marker and the second marker has to be a substance whose optical characteristic is changed depending on whether or not the first nucleic acid molecule probe and the second nucleic acid molecule probe are hybridized.

Here, the phrase “substance whose optical characteristic is changed” means that the wavelength or the intensity of fluorescence emitting from the substance is changed. Moreover, in the present invention, the phrase “to detect an optical characteristic of a marker” means to detect a fluorescence signal of a specific wavelength emitting from the marker. Examples of such a fluorescence signal can include fluorescence intensity and fluorescence polarization. In the present invention, the fluorescence signal is preferably fluorescence intensity.

That is, when the first nucleic acid molecule probe conjugated with the first marker and the second nucleic acid molecule probe conjugated with the second marker are hybridized, FRET energy transfer will occur between the first marker and the second marker. On the other hand, when both parties are not hybridized and dissociated, no FRET will occur between the first marker and the second marker. For this reason, the optical characteristic of at least one of the first marker and the second marker is changed depending on whether or not the first nucleic acid molecule probe and the second nucleic acid molecule probe are hybridized. Here, in a sample solution containing the first nucleic acid molecule probe, the second nucleic acid molecule probe, and the target nucleic acid molecule, the first nucleic acid molecule probe which does not hybridize with the second nucleic acid molecule probe can be assumed to hybridize with the target nucleic acid molecule. Therefore, the target nucleic acid molecule can be detected through detection of a time course change in the optical characteristic of the first marker or the second marker.

In the present invention, the first marker and the second marker may be any substances as long as they are in a combination which can induce FRET when they come sufficiently close to each other, that is, when the first nucleic acid molecule probe and the second marker are hybridized. The first marker and the second marker can be used by appropriate selection among usual substances for use in FRET. For example, both of the first marker and the second marker may be fluorescent dyes, or either one of them may be a fluorescent dye and the other one may be a substance which can radiate the energy received from the fluorescent dye in a form of heat energy (a so-called dark quencher).

In addition, in the target nucleic acid molecule quantification method of the present invention, either one of the first marker and the second marker may be a fluorescent substance. That is, the first marker may be a fluorescent substance and the second marker may be a quenching substance for quenching fluorescence emitting from the fluorescent substance, or the second marker may be a fluorescent substance and the first marker may be a quenching substance for quenching fluorescence emitting from the fluorescent substance.

In the claims and the description of this application, the term “fluorescent dye” refers to a dye which has a property to emit fluorescence. On the other hand, the term “fluorescent substance” refers to a substance to serve as a FRET donor and the term “quenching substance” refers to a substance to serve as a FRET acceptor.

The first nucleic acid molecule probe and the second nucleic acid molecule probe are produced by designing their nucleotide sequences on the basis of the nucleotide sequence data of the target nucleic acid molecule, and conjugating markers to the thus synthesized nucleic acid molecule probes. The design and the synthesis of these nucleic acid molecule probes and the reaction for conjugating the markers to the nucleic acid molecule probes can be conducted by usual methods.

Moreover, the target nucleic acid molecule quantification method of the present invention comprises: forming a hybrid between the target nucleic acid molecule and the first nucleic acid molecule probe under a condition where both parties can be specifically hybridized, and any nonspecific hybridization between the first nucleic acid molecule probe and another nucleic acid comprising a nucleotide sequence that is not complementary to the first nucleic acid molecule probe can be sufficiently suppressed (hereunder, referred to as the “specific hybridization condition”); then forming a covalent bond between two nucleic acid strands in the thus formed hybrid (between the first nucleic acid molecule probe and the target nucleic acid molecule) under the same condition so as to stabilize the hybrid between the target nucleic acid molecule and the first nucleic acid molecule; and then detecting the concerned hybrid.

As described above, in the present invention, the hybrid between the target nucleic acid molecule and the first nucleic acid molecule probe is to be detected by utilizing FRET. However, the measurement of FRET under the specific hybridization condition is difficult and requires an expensive detection device. For this reason, a produced hybrid is generally detected under room temperature or such a comparatively moderate condition where a nonspecific hybrid between the first nucleic acid molecule probe and a nucleic acid comprising a nucleotide sequence that is not complementary to the first nucleic acid molecule probe can also be formed in a similar fashion to the hybrid between the target nucleic acid molecule and the first nucleic acid molecule probe (hereunder, referred to as the “nonspecific hybridization condition”). In this nonspecific hybridization condition, the hybrid between the target nucleic acid molecule and the first nucleic acid molecule probe can be replaced by a hybrid between the second nucleic acid molecule probe and the first nucleic acid molecule probe, or by a hybrid between the first nucleic acid molecule probe and a nucleic acid comprising a nucleotide sequence that is not complementary to the first nucleic acid molecule probe. That is, non-target nucleic acid molecules may be sometimes detected by error through the FRET measurement under a nonspecific hybridization condition. On the other hand, in the target nucleic acid molecule quantification method of the present invention, the hybrid between the target nucleic acid molecule and the first nucleic acid molecule probe that has been specifically formed under a specific hybridization condition can be stably maintained until the time of the FRET detection, and therefore the specificity and the quantitativity of the detection for the target nucleic acid molecule can be remarkably improved.

The specific hybridization condition is dependent on the types and the lengths of the nucleotide sequences of the target nucleic acid molecule, the first nucleic acid molecule probe, and the second nucleic acid molecule probe. Specifically, the specific hybridization condition between the target nucleic acid molecule and the first nucleic acid molecule probe can be determined from a melting curve of the first nucleic acid molecule probe.

The hybrid formation is generally dependent on the temperature condition and the salt concentration condition. Therefore, the melting curve can be obtained by measuring the absorbance or the fluorescence intensity of the solution which contains only the first nucleic acid molecule probe and the second nucleic acid molecule probe, while varying the temperature of the solution from high temperature to low temperature. From the thus obtained melting curve, the temperature condition ranging from the temperature at which the first nucleic acid molecule probe and the second nucleic acid molecule probe that have been single-stranded by denaturation, start to form a hybrid with each other, up to the temperature at which almost all probes are hybridized, can be set as the specific hybridization condition. The melting curve can also be obtained by determining the specific hybridization condition not by varying the temperature but instead by varying the salt concentration in the solution from low concentration to high concentration in the same manner.

In this manner, the specific hybridization condition differs depending on the types of the target nucleic acid molecule and the first nucleic acid molecule probe, and can be experimentally determined. However, generally speaking, the Tm value (melting temperature) can be substituted for the experimental determination. For example, the Tm value (temperature at which 50% of double-stranded DNA dissociates into single-stranded DNAs) can be calculated from the nucleotide sequence data of the first nucleic acid molecule probe with use of a universal primer/probe design software, or the like. A condition having its temperature close to the Tm value, for example, approximately within the Tm value±3° C., can be set as the specific hybridization condition. The specific hybridization condition can be determined in more detail by experimentally obtaining the melting curve around the calculated Tm value.

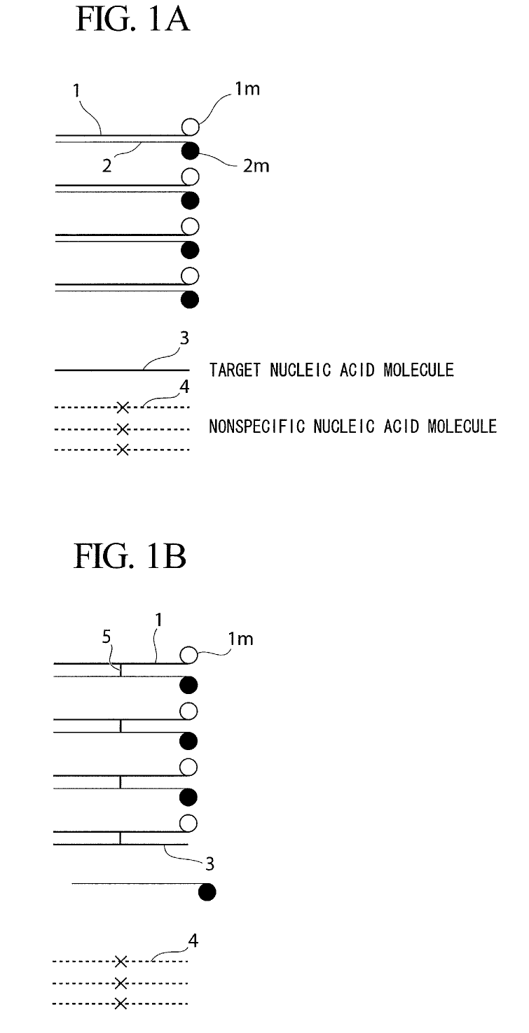

FIG. 1 is a schematic diagram showing the effect of covalent bond formation (crosslink) between the target nucleic acid molecule and the first nucleic acid molecule probe which constitute a hybrid. FIG. 1 (A) shows the target nucleic acid molecule (3), a nonspecific nucleic acid molecule (4), the first nucleic acid molecule probe (1) conjugated with the first marker (1m), and the second nucleic acid molecule probe (2) conjugated with the second marker (2m), in the sample solution before the hybrid formation. The nonspecific nucleic acid molecule (4) refers to a nucleic acid molecule comprising a nucleotide sequence that is not complementary to the first nucleic acid molecule probe. In the drawing, the symbol “x” refers to a nucleotide which is not complementary to the first nucleic acid molecule probe. Usually, before the denaturation step, the first nucleic acid molecule probe (1) and the second nucleic acid molecule probe (2) are forming a hybrid. On the other hand, FIG. 1 (B) shows a state after the sample solution of FIG. 1 (A) has been denatured, the hybridization has been performed at an optimized temperature for the hybridization between the first nucleic acid molecule probe (1) and the target nucleic acid molecule (3) (specific hybridization condition), and the sample solution has been irradiated with light while keeping the temperature of the sample solution at the concerned temperature, to thereby form covalent bonds (5) respectively between the first nucleic acid molecule probe (1) and the target nucleic acid molecule (3), and between the first nucleic acid molecule probe (1) and the second nucleic acid molecule probe (2), through a photochemical reaction that will be described later. In the case where the covalent bonds are not formed, when the temperature of the sample solution is lowered to about a room temperature on completion of the hybrid formation treatment, the nonspecific nucleic acid molecule (4) is substituted with the target nucleic acid molecule (3) and thereby a hybrid between the nonspecific nucleic acid molecule (4) and the first nucleic acid molecule probe (1) is formed. However, in the present invention, since the hybrid has been stabilized by forming a covalent bond in advance before lowering the temperature of the sample solution to room temperature, the state at the completion of the hybrid formation treatment as shown in FIG. 1 (B) can be maintained even at the time of the hybrid detection with the FRET measurement or the like, and therefore the number of the target nucleic acid molecules that are specifically hybridizing with the first nucleic acid molecule probe can be subjected to the quantitative analysis.

In this manner, in the target nucleic acid molecule quantification method of the present invention, since the hybrid formed by the hybridization reaction can be stabilized in the state at the time of the hybrid formation under the specific hybridization condition, the hybridization reaction can be detected with high specificity. Therefore, with use of the target nucleic acid molecule quantification method of the present invention, it also becomes possible to specifically and exclusively detect the target nucleic acid molecule that is completely matched to the first nucleic acid molecule probe in the nucleic acid-containing sample which mixingly contains the target nucleic acid molecule and nucleic acid molecules having mismatch sites differing from this molecule.

The target nucleic acid molecule quantification method of the present invention specifically comprises the following (a) to (e): (a) preparing a sample solution comprising a nucleic acid-containing sample, a first nucleic acid molecule probe conjugated with a first marker, and a second nucleic acid molecule probe conjugated with a second marker; (b) denaturing nucleic acid molecules in the sample solution that has been prepared in (a); (c) hybridizing the nucleic acid molecules in the sample solution, after (b); (d) forming a covalent bond between two nucleic acid strands in the thus formed hybrid under a same condition, regarding the temperature and the salt concentration of the sample solution, as that of the hybrid formation in (c), after (c); and (e) quantifying the target nucleic acid molecules by detecting a time course change in an optical characteristic of the first marker or the second marker in the sample solution, after (d).

In the present invention, the term “target nucleic acid molecule” refers to a nucleic acid molecule which comprises a certain specific nucleotide sequence serving as the target of detection and quantification. The target nucleic acid molecule is not specifically limited as long as its nucleotide sequence has been elucidated to an extent that allows a design of the first nucleic acid molecule probe and the like. For example, the target nucleic acid molecule may be a nucleic acid molecule which comprises a nucleotide sequence found in an animal or plant chromosome or a nucleotide sequence found in a bacterial or viral gene, or a nucleic acid molecule which comprises an artificially designed nucleotide sequence. In the present invention, the term “target nucleic acid molecule” may be either a double-stranded nucleic acid or a single-stranded nucleic acid. In addition, the term “target nucleic acid molecule” may be either DNA or RNA. Examples of the target nucleic acid molecule can include a micro RNA, an siRNA, an mRNA, a hnRNA, a genomic DNA, a DNA synthesized by PCR amplification or the like, and a cDNA synthesized from RNA with use of a reverse transcriptase. In the target nucleic acid molecule quantification method of the present invention, a micro RNA or an siRNA is preferred. In the present invention, the length of the target nucleic acid molecule is not specifically limited, although it is preferably 10 nucleotides or longer, more preferably about 10 to 500 nucleotides, and yet more preferably about 10 to 50 nucleotides. Of these, a micro RNA or an siRNA in a length of about 10 to 30 nucleotides is particularly preferred.

In addition, in the present invention, the nucleic acid-containing sample is not specifically limited as long as the sample contains nucleic acid molecules. Examples of the nucleic acid-containing sample can include a biological sample collected from an animal or the like, a sample prepared from cultured cells or the like, and a reaction solution on completion of a nucleic acid synthesis reaction. The sample may be in an intact form such as a biological sample per se, or a processed form such as a nucleic acid solution extracted and purified from a biological sample.

First, as (a), the sample solution is prepared by adding the nucleic acid-containing sample, the first nucleic acid molecule probe, and the second nucleic acid molecule probe, to an appropriate solvent. The solvent is not specifically limited as long as it does not inhibit the detection of fluorescence emitting from the first marker or the second marker, nor FRET occurring between both markers. The selection of the solvent can be appropriately made from buffers for general use in the technical field. Examples of such buffers can include phosphate buffers such as PBS (phosphate buffered saline at pH 7.4) and Tris buffers.

Generally speaking, if the preparation of the sample solution in (a) is carried out at a temperature equal to or lower than the Tm value of the first nucleic acid molecule probe and the second nucleic acid molecule probe, both probes are hybridized and stay in a state of double-stranded nucleic acids. For this reason, if a fluorescent substance is used as the first marker and a quenching substance is used as the second marker, fluorescence emitting from the first marker is quenched by FRET occurring between the first marker and the second marker, and thus it becomes undetectable or attenuated.

Next, as (b), nucleic acid molecules in the prepared sample solution are denatured. In the present invention, the phrase “a nucleic acid molecule is denatured” means to dissociate a double-stranded nucleic acid into single-stranded nucleic acids. In the present invention, the denaturation is preferably performed by a high temperature treatment (heat denaturation) or a treatment with a low salt concentration solution because such treatments will impose relatively less influence on the fluorescent substance. Of these, heat denaturation is preferred because the operation is easy. Specifically, in the heat denaturation, the nucleic acid molecules in the sample solution can be denatured by subjecting the sample solution to a high temperature treatment (for example, 90° C. or higher). By this denaturation, the respective nucleic acid molecule probes in the sample solution are brought into a dissociated single-stranded state. In addition, if the target nucleic acid molecule is a double-stranded nucleic acid, the target nucleic acid molecule also dissociates into single-stranded nucleic acids in the same manner. On the other hand, the denaturation by means of a low salt concentration treatment can be conducted through adjustment of the salt concentration of the sample solution at a sufficiently low degree by, for example, dilution with purified water.

Next, as (c), the nucleic acid molecules in the sample solution are hybridized. If the heat denaturation has been performed, the nucleic acid molecules in the sample solution can be appropriately hybridized by lowering the temperature of the sample solution to a suitable temperature for the specific hybridization condition, on completion of the high temperature treatment. Preferably, the temperature of the sample solution is lowered to approximately within the Tm value of the first nucleic acid molecule probe ±3° C. On the other hand, if the denaturation has been performed by the low salt concentration treatment, the nucleic acid molecules in the sample solution can be appropriately hybridized in the same manner by increasing the salt concentration of the sample solution to a suitable concentration for the specific hybridization condition through addition with a salt solution or such adjustment, on completion of the low salt concentration treatment.

Through these treatments, the first nucleic acid molecule probe is hybridized. However, at this time, the first nucleic acid molecule probe can hybridize to either the second nucleic acid molecule probe or the target nucleic acid molecule. Therefore, if the target nucleic acid molecule is present in the nucleic acid-containing sample, the hybrid between the first nucleic acid molecule probe and the target nucleic acid molecule and the hybrid between the first nucleic acid molecule probe and the second nucleic acid molecule probe are both present in the sample solution after the step (c).

Thereafter, as (d), a covalent bond is formed between two nucleic acid strands in the thus formed hybrid under the same condition, in terms of the temperature and the salt concentration of the sample solution, as that of the hybrid formation in (c), that is, the specific hybridization condition. The term “the same condition as that of the hybrid formation in (c)” is preferably a condition in which both the temperature and the salt concentration of the sample solution are the same. However, the condition may not always have to be completely and physically the same so long as the readiness of the hybrid formation between the target nucleic acid molecule and the first nucleic acid molecule probe and the readiness of the hybrid formation between the first nucleic acid molecule probe and another nucleic acid comprising a nucleotide sequence that is not complementary to the first nucleic acid molecule probe are substantially the same at the time of the hybrid formation in (c) and at the time of the covalent bond formation in (d). For example, if the temperature of the sample solution at the time of the hybrid formation in the step (c) is within the Tm value±3° C., it may be sometimes possible to change the condition as long as the temperature of the sample solution at the time of the covalent bond formation in (d) remains within the Tm value±3° C. The reason is that any condition satisfying the temperature within the Tm value±3° C. may be able to meet the criteria for the specific hybridization condition for some types of the nucleotide sequence of the target nucleic acid molecule, and the condition fluctuation within this temperature range can be deemed to impose almost no influence on the specificity for the hybrid formation.

The method for forming a covalent bond in (d) is not specifically limited as long as a covalent bond can be formed between two strands within the hybrid formed by hybridization between the single-stranded target nucleic acid molecule and the single-stranded first nucleic acid molecule probe, and any technique for use for crosslinking between nucleic acid molecules may be employed. For example, it is possible to employ either a method for selectively and exclusively forming a covalent bond in the hybrid between the target nucleic acid molecule and the first nucleic acid molecule probe, or a method for forming a covalent bond not only in the concerned hybrid but also in another hybrid existing in the sample solution after (c).

In the present invention, it is preferable to form a covalent bond through a photochemical reaction. The term “photochemical reaction” refers to a reaction led by irradiation with light at a specific wavelength and carried on by utilizing the light energy thereof. The covalent bond formation method by means of a photochemical reaction is capable of forming a covalent bond between nucleic acid strands within a hybrid by irradiating the sample solution with light at a specific wavelength, and thus it is not necessary to change the condition such as the composition of the sample solution. For this reason, it is possible to suppress other influences on hybrids in the sample solution except for the covalent bond formation, and to facilitate the operation.

For example, when using a kind of the first nucleic acid molecule probe at least one nucleotide of which in the nucleotide sequence complementary to the target nucleic acid molecule is substituted by a photoreactive nucleotide derivative, a covalent bond can be formed via the photoreactive nucleotide derivative in the hybrid between the target nucleic acid molecule and the first nucleic acid molecule probe through the photochemical reaction. Here, the term “photoreactive nucleotide derivative” refers to a nucleotide derivative which has a site whose reactivity for an organic synthetic reaction can be activated by irradiation with light at a specific wavelength (photoreactive site) as well as being capable of forming a nucleic acid strand in the same manner as for natural nucleotides. On completion of the hybrid formation between the target nucleic acid molecule and the first nucleic acid molecule probe substituted with the photoreactive nucleotide derivative, when irradiating the sample solution containing this hybrid with light at a wavelength capable of activating the photoreactive site in the photoreactive nucleotide derivative, the photoreactive site is activated and thereby a covalent bond is formed between this photoreactive site and a nearby atom in the target nucleic acid molecule.

Examples of such a photoreactive nucleotide derivative can include 3-cyanovinylcarbazole nucleoside (^{CNV}K) (for example, refer to Non-patent Document 4 or 5). The first nucleic acid molecule probe substituted with the photoreactive nucleotide derivative can be produced, for example, by using a photoreactive nucleotide derivative as a raw ingredient when synthesizing the first nucleic acid molecule probe with a known oligonucleotide synthesizer. In addition, it is also possible to obtain this kind of the first nucleic acid molecule probe by producing an unsubstituted first nucleic acid molecule probe and thereafter introducing an appropriate photoreactive functional group into a component nucleotide of the probe, through a known organic synthetic reaction.

If ^{CNV}K is used as a photoreactive nucleotide derivative, the hybrid between the target nucleic acid molecule and the first nucleic acid molecule probe can be stabilized by the covalent bond specifically in the following manner. First, among nucleotides of the first nucleic acid molecule probe which can form base pairs with the target nucleic acid molecule, at least one nucleotide whose adjacent nucleotide on the 5′ side is a purine nucleotide is substituted by ^{CNV}K to prepare a ^{CNV}K-substituted first nucleic acid molecule probe. Next, this ^{CNV}K-substituted first nucleic acid molecule probe and the target nucleic acid molecule are hybridized, and thereafter the sample solution containing the thus formed hybrid is irradiated with light at 340 to 380 nm, preferably ultraviolet light including 366 nm. Then, a component atom of the ^{CNV}K and a component atom of a pyrimidine nucleotide of the target nucleic acid molecule that fauns a base pair with the purine nucleotide adjacent to the 5′ side of the ^{CNV}K are bound by a covalent bond.

Besides, it is also possible to use a kind of the first nucleic acid molecule probe having psoralen, as a photoreactive nucleotide derivative, attached to thymine (T) or adenine (A) via a linker (for example, refer to Proc. Natl. Acad. Sci. USA, Vol. 88, pp. 5602-5606, July 1991). For example, in the case where a TA sequence is present in a region of the first nucleic acid molecule probe to hybridize with the target nucleic acid molecule, a psoralen-conjugated first nucleic acid molecule probe having psoralen attached to T or A in the TA sequence via a linker is prepared. Next, this psoralen-conjugated first nucleic acid molecule probe and the target nucleic acid molecule are hybridized, and then this hybrid is irradiated with near-ultraviolet light of 254 nm or the like. By so doing, the target nucleic acid molecule and the psoralen-conjugated first nucleic acid molecule probe are crosslinked via this psoralen, and the hybrid between them can be stabilized.

Furthermore, as (e), the target nucleic acid molecules can be quantified by detecting a time course change in an optical characteristic of the first marker or the second marker in the sample solution. For example, the first nucleic acid molecule probe hybridizing with the second nucleic acid molecule probe is in a quenched state, whereas the first nucleic acid molecule probe hybridizing with the target nucleic acid molecule is not in a quenched state led by fluorescence energy transfer since there is no quenching substance in near positions, because of which fluorescence emitting from the first marker is detected. Accordingly, by irradiating with light at an optimum wavelength for the spectral characteristic of the first marker, and measuring the fluctuation (time course change) of the intensity of fluorescence emitting from the first marker, it becomes possible to detect whether the first marker in the sample solution is a molecule hybridizing with the target nucleic acid molecule or a molecule hybridizing with the second nucleic acid molecule probe, for individual molecules so as to thereby quantify the target nucleic acid molecules.

In (e), the method for detecting the time course change in an optical characteristic of the first marker or the second marker in the sample solution is not specifically limited as long as this method is capable of detecting and analyzing the time course change (fluctuation) of the fluorescence signal from molecules in the solution. For example, the time course change of the fluorescence signal from molecules in the solution can be measured and analyzed by detection and analysis of the fluorescence signal from molecules existing in the focal area of a confocal optical system. Examples of such a method can include fluorescence intensity distribution analysis (FIDA), fluorescence correlation spectroscopy (FCS), fluorescence cross-correlation spectroscopy (FCCS), and FIDA polarization (FIDA-PO). Such detection and analysis of the time course change of the fluorescence signal from molecules can be carried out with use of, for example, a known single molecule fluorescence spectroscopy system such as MF20 (manufactured by Olympus).

In the target nucleic acid molecule quantification method of the present invention, since the change in the optical characteristic caused by FRET is to be detected, the method for detecting and analyzing the time course change in the optical characteristic of each marker is preferably FIDA, FCS, or FCCS for analyzing the fluorescence intensity, more preferably FIDA or FCS, and yet more preferably FIDA.

In addition, in these single molecule fluorescence spectroscopy methods, through detection of the fluorescence signal from respective molecules in the sample solution and a statistical analysis thereof, nucleic acid molecule probes involving FRET and nucleic acid molecule probes not involving FRET can be distinguished. Here, the number of the first nucleic acid molecule probes not involving FRET is theoretically equal to the number of the first nucleic acid molecule probes hybridizing with the target nucleic acid molecules. Therefore, the number of the target nucleic acid molecules can be directly measured.

For example, the target nucleic acid molecules can be quantified by calculating the number of molecules of the first nucleic acid molecule probes hybridizing with the target nucleic acid molecules, through detection of the fluctuation of the fluorescence intensity of molecules existing in the focal area of a confocal optical system by FIDA and subsequent statistical analysis. Moreover, if the target nucleic acid molecule is a double-stranded nucleic acid, one strand out of the two strands made by dissociation from the target nucleic acid molecule will hybridize with the first nucleic acid molecule probe and the other one strand will hybridize with the second nucleic acid molecule probe. For this reason, the target nucleic acid molecules can also be quantified by calculating the number of molecules of the second nucleic acid molecule probes hybridizing with the target nucleic acid molecules.

The target nucleic acid molecules can also be quantified in the same manner by calculating the number of molecules of the first nucleic acid molecule probes or the second nucleic acid molecule probes hybridizing with the target nucleic acid molecules, through detection of the fluctuation of the fluorescence intensity of molecules existing in the focal area of a confocal optical system by autocorrelation analysis (FCS) or fluorescence cross-correlation spectroscopy (FCCS) and subsequent statistical analysis. In the case of FCS or FCCS, the target nucleic acid molecules can be more precisely quantified by using a dark quencher as a quenching substance.

In this way, the target nucleic acid molecule quantification method of the present invention is a method for directly and precisely detecting the hybridization between a target nucleic acid molecule and a nucleic acid molecule probe through detection and analysis of a time course change in an optical characteristic such as the fluorescence intensity caused by so-called FRET energy transfer for individual molecules, with use of a single molecule fluorescence spectroscopy method such as FIDA and FCS, and thus this method is capable of quantification of the concentration of the target nucleic acid molecules.

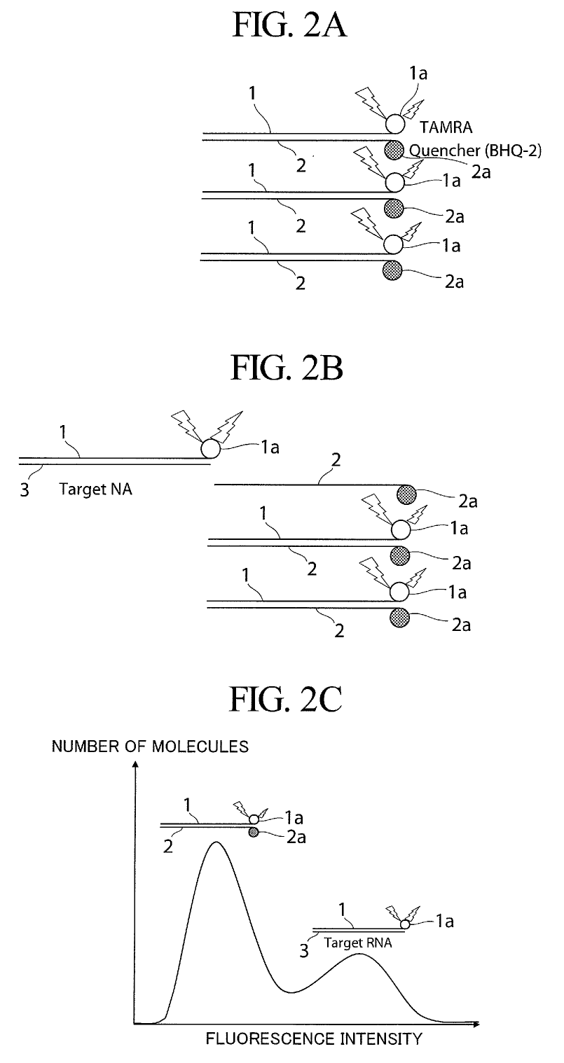

FIG. 2 shows one aspect of the target nucleic acid molecule quantification method of the present invention in which a fluorescent dye is used as the first marker and a dark quencher is used as the second marker. FIG. 2 (A) shows a state before the preparation of the sample solution, that is, a state before the addition of the target nucleic acid molecule (target nucleic acid, Target NA) (3) in which the first nucleic acid molecule probe (1) conjugated with a first marker fluorescent dye TAMRA (1a) and the second nucleic acid molecule probe (2) conjugated with a second marker BHQ-2 (2a) serving as a quenching substance of TAMRA are hybridized with each other. On the other hand, FIG. 2 (B) shows a state of hybrids after the addition of the nucleic acid-containing sample which contains the target nucleic acid molecule (target nucleic acid, denoted by “Target NA” in the drawing) (3) to these nucleic acid molecule probes, followed by mixing the solution, denaturation, and hybridization. Some of the first nucleic acid molecule probes (1) are hybridized with the target nucleic acid molecules (3) while the remaining first nucleic acid molecule probes (1) are re-hybridized with the second nucleic acid molecule probes (2). The second nucleic acid molecule probes (2) which are not re-hybridized with the first nucleic acid molecule probes (1) do not emit fluorescence because the second marker BHQ-2 (2a) is a dark quencher. On the other hand, the first nucleic acid molecule probes (1) which are hybridized with the target nucleic acid molecules (3) emit fluorescence because there is no quenching substance in near positions. For this reason, by measuring this sample solution by FIDA and subjecting it to the statistical analysis, it becomes possible to measure these hybrids as two types of fluorescent molecules having different fluorescence intensities as shown in FIG. 2 (C). FIG. 2 (C) shows the distribution of molecules at respective fluorescence intensities in the sample solution, resulting from the analysis. The molecules at low fluorescence intensities represent a hybrid molecule between the first nucleic acid molecule probe (1) and the second nucleic acid molecule probe (2), and the molecules at high fluorescence intensities represent a hybrid molecule between the first nucleic acid molecule probe (1) and the target nucleic acid molecule (3). That is, the number of molecules at high fluorescence intensities is the number of the target nucleic acid molecules (3), and therefore the target nucleic acid molecule can be quantified.

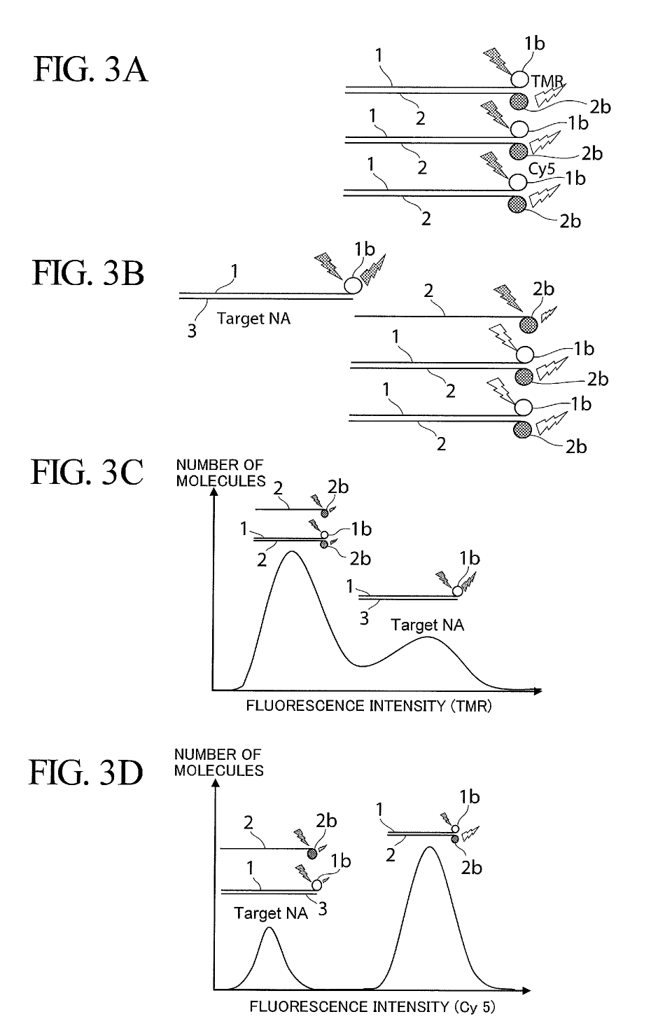

FIG. 3 shows another aspect of the target nucleic acid molecule quantification method of the present invention in which fluorescent dyes are used as the first marker and the second marker. FIG. 3 (A) shows a state before the preparation of the sample solution, that is, a state before the addition of the target nucleic acid molecule (target nucleic acid, Target NA) (3) in which the first nucleic acid molecule probe (1) conjugated with a first marker fluorescent dye Cy5 [TMR] (1b) and the second nucleic acid molecule probe (2) conjugated with a second marker fluorescent dye TAIVIRA [Cy5] (2b) serving as a quenching substance of Cy5 [TMR] are hybridized with each other. In the aspect shown in FIG. 2, the hybrid between the first nucleic acid molecule probe and the second nucleic acid molecule probe does not emit fluorescence, whereas in the aspect shown in FIG. 3, when both parties are hybridized, the second marker Cy5 (2b) and the first marker TMR (1b) induce fluorescence energy resonance and the second marker Cy5 (2b) is excited at the excitation wavelength of the fluorescent molecule of the first marker TMR (1b). Therefore, by irradiating with the excitation wavelength of the first marker and detecting the fluorescence from the second marker, it can be understood that the first marker and the second marker are positioned close to each other, that is, the first nucleic acid molecule probe and the second nucleic acid molecule probe are hybridized. On the other hand, FIG. 3 (B) shows a state of hybrids after the addition of the nucleic acid-containing sample which contains the target nucleic acid molecule (target nucleic acid, denoted by “Target NA” in the drawing) (3) to these nucleic acid molecule probes, followed by mixing the solution, denaturation, and hybridization. Some parts of the first nucleic acid molecule probes (1) are hybridized with the target nucleic acid molecules (3) while the remaining first nucleic acid molecule probes (1) are re-hybridized with the second nucleic acid molecule probes (2). FIG. 3 (C) shows the distribution of molecules at respective fluorescence intensities in the sample solution, resulting from the excitation of this sample solution with light at 543 nm serving as the excitation wavelength of TMR, the detection for light of the fluorescence wavelength of TMR, and the statistical analysis by FIDA. In this case, a strong fluorescence signal is observed from the first nucleic acid molecule probe (1) hybridizing with the target nucleic acid molecule (3), whereas a weak fluorescence signal is observed from the first nucleic acid molecule probe (1) hybridizing with the second nucleic acid molecule probe (2), or the second nucleic acid molecule probe (2) alone. That is, the number of molecules at high fluorescence intensities is the number of the target nucleic acid molecules (3). On the other hand, FIG. 3 (D) shows the distribution of molecules at respective fluorescence intensities in the sample solution, resulting from the excitation of this sample solution with light at 543 nm serving as the excitation wavelength of TMR, the detection for light of the fluorescence wavelength of Cy5 (670 nm), and the statistical analysis by FIDA. In this case, isolated and detected are molecules emitting a strong fluorescence signal by fluorescence energy transfer only from the hybrid between the first nucleic acid molecule probe (1) hybridizing with the second nucleic acid molecule probe (2). That is, the detection for light of the fluorescence wavelength of Cy5 can ensure the competitive reaction which can be found by detection for light of the fluorescence wavelength of TMR, and therefore the preciseness can be improved.

Moreover, it is possible for the target nucleic acid molecule quantification method of the present invention to more readily quantify target nucleic acid molecules in a nucleic acid-containing sample by using a kit including the first nucleic acid molecule probe and the second nucleic acid molecule probe mentioned above. In particular, the kit preferably includes a kind of the first nucleic acid molecule probe at least one nucleotide of which in the nucleotide sequence complementary to the target nucleic acid molecule is substituted by a photoreactive nucleotide derivative. The kit may include other reagents such as a buffer for use in the preparation of the sample solution, or the like, in addition to these nucleic acid molecule probes.

### EXAMPLES

Next is a more detailed description of the present invention with reference to examples. However, the present invention is not to be limited by the examples below.

### Example 1

The concentration of non-labeled target nucleic acid molecules was efficiently measured by the target nucleic acid molecule quantification method of the present invention when a single-stranded DNA comprising a sequence homologous with a human micro RNA, miR-21 (hsa-miR-21, 5′-UAGCUUAUCAGACUGAUGUUGA-3′, the miRBase Sequence Database Release 12.0, http://microrna.sanger.ac.uk/sequences/index.shtml) was set as the target nucleic acid molecule.

First, a first nucleic acid molecule probe comprising a nucleotide sequence complementary to miR-21, TAMRA as a first marker conjugated to the 5′ end, and one nucleotide substituted by 3-cyanovinylcarbazole nucleoside (^{CNV}K) as a crosslinking nucleotide derivative, and a second nucleic acid molecule probe comprising a nucleotide sequence homologous with the first nucleic acid molecule probe and BHQ-2 as a second marker conjugated to the 5′ end, were prepared. The respective probes were added at 10 nM each in a solution. The solution was mixed and added with the target nucleic acid molecule (single-stranded DNA comprising a sequence homologous to miR-21) at final concentrations of 0.08 to 10 nM to thereby prepare respective sample solutions. The respective sample solutions were subjected to denaturation at 90° C. for 10 minutes, hybridization by lowering the temperature at a rate of 1° C./minute until 62° C., and irradiation with light at 366 nm (crosslinking) at 62° C. Then, the solutions were cooled down to a room temperature, followed by irradiation with light at the excitation wavelength of TAMRA, detection for light of the fluorescence wavelength of TAMRA, and FIDA analysis.

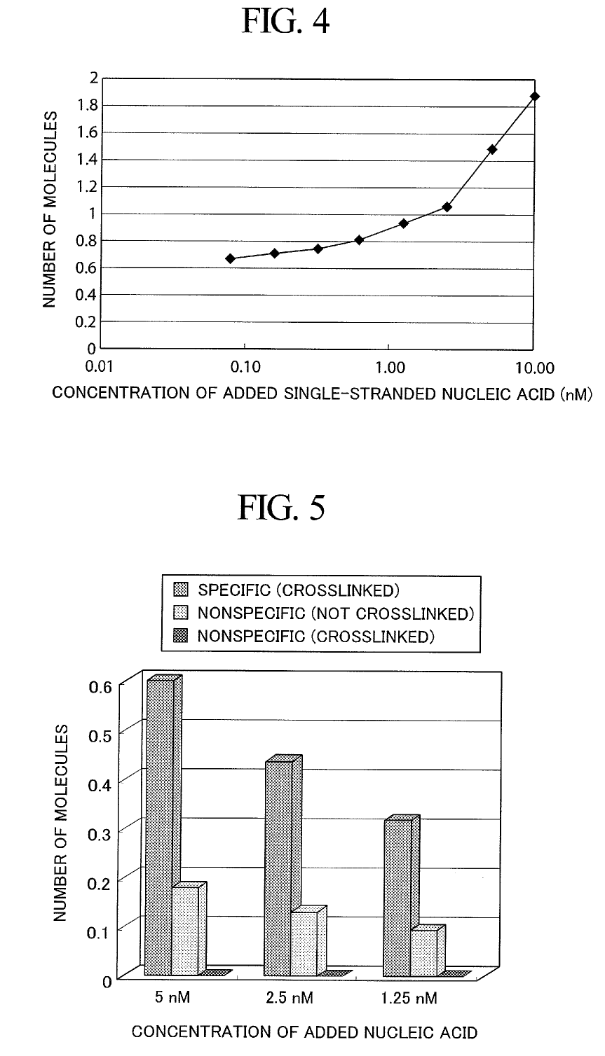

The obtained analysis results are shown in FIG. 4. The x-axis indicates the concentration of the added single-stranded DNA (target nucleic acid molecule), and the y-axis indicates the number of molecules from which the fluorescence of TAMRA was detected in the confocal area, that is, the number of the first nucleic acid molecule probes whose fluorescence had been recovered as a result of hybridization with the added single-stranded DNA instead of the second nucleic acid molecule probe. The number of the first nucleic acid molecule probes whose fluorescence had been recovered was increased in proportion with the concentration of the added single-stranded DNA.

From these results, it is apparently possible to quantify the target nucleic acid molecules existing in the sample solution by using the target nucleic acid molecule quantification method of the present invention. The added nucleic acid in this example (target nucleic acid molecule) was DNA, although it is possible to use RNA in the same manner

### Example 2

Next, in order to verify the specificity, two types of nucleic acids, that is, the single-stranded DNA comprising a sequence homologous to miR-21 that had been used in Example 1 (specific nucleic acid), and a single-stranded DNA in which one nucleotide of the specific nucleic acid had been substituted from C (cytosine) to G (guanine) (nonspecific nucleic acid), were respectively quantified by the target nucleic acid molecule quantification method of the present invention. In addition, in order to verify the effect of covalent bond formation, a probe comprising the identical nucleotide sequence to the first nucleic acid molecule probe used in Example 1 and TAMRA as a first marker conjugated to the 5′ end (without the introduction of ^{CNV}K) was prepared as an unsubstituted first nucleic acid molecule probe, in addition to the first nucleic acid molecule probe and the second nucleic acid molecule probe used in Example 1.

Specifically, the first nucleic acid molecule probe and the second nucleic acid molecule probe were added at 10 nM each in a solution. The solution was mixed and added with the specific nucleic acid or the nonspecific nucleic acid at final concentrations of 1.25 nM, 2.5 nM, and 5 nM to thereby prepare respective sample solutions. Furthermore, the unsubstituted first nucleic acid molecule probe and the second nucleic acid molecule probe were added at 10 nM each in a solution. The solution was mixed and added with the nonspecific nucleic acid at final concentrations of 1.25 nM, 2.5 nM, and 5 nM to thereby prepare respective sample solutions. The respective sample solutions were subjected to denaturation, hybridization, and irradiation with light at 366 nm (crosslinking) at 62° C. in the same manner as that of Example 1. Then, the solutions were cooled down to a room temperature, followed by irradiation with light at the excitation wavelength of TAMRA, detection for light of the fluorescence wavelength of TAMRA, and FIDA analysis.

The obtained analysis results are shown in FIG. 5. The x-axis and the y-axis indicate the same meanings as those of FIG. 4. In the graph, the caption “Specific (crosslinked)” shows the results of the sample solutions containing the specific nucleic acid and the ^{CNV}K-substituted first nucleic acid molecule probe, the caption “Nonspecific (crosslinked)” shows the results of the sample solutions containing the nonspecific nucleic acid and the ^{CNV}K-substituted first nucleic acid molecule probe, and the caption “Nonspecific (not crosslinked)” shows the results of the sample solutions containing the nonspecific nucleic acid and the unsubstituted first nucleic acid molecule probe. In these results, among the sample solutions containing the nonspecific nucleic acid, no fluorescence signal of TAMRA was detected in the solutions applied with the crosslinking treatment (irradiation with light at 366 nm) with use of the ^{CNV}K-substituted first nucleic acid molecule probe, showing no presence of the first nucleic acid molecule probe hybridizing with the nonspecific nucleic acid instead of the second nucleic acid molecule probe. On the other hand, among the sample solutions containing the nonspecific nucleic acid, fluorescence signals of TAMRA were detected in the solutions with the unsubstituted first nucleic acid molecule probe, showing the presence of the first nucleic acid molecule probe hybridizing with the nonspecific nucleic acid.

There is one nucleotide mismatch between the first nucleic acid molecule probe and the nonspecific nucleic acid. Therefore, the fluorescence signals of TAMRA detected in the sample solutions containing the nonspecific nucleic acid are nonspecific signals. From these results, it is apparently possible to remarkably suppress the formation of nonspecific hybrids through previous covalent bond formation between two nucleic acid strands in a hybrid under a specific hybridization condition, that is, it is possible to identify even one nucleotide mismatch or such a tiny mismatch and to specifically detect the target nucleic acid molecule by using the target nucleic acid molecule quantification method of the present invention.

### INDUSTRIAL APPLICABILITY

The target nucleic acid molecule quantification method of the present invention is capable of highly sensitive and precise quantification of target nucleic acid molecules existing in a sample through detection and analysis of FRET energy transfer for individual molecules, and thus is applicable to the fields of biochemistry, molecular biology, clinical tests, and the like, in which nucleic acids in a sample are to be subjected to quantitative analysis.

### BRIEF DESCRIPTION OF THE REFERENCE SYMBOLS

1: First nucleic acid molecule probe, 1m: First marker, 1a: First marker (TAMRA), 1b: First marker (TMR), 2: Second nucleic acid molecule probe, 2m: Second marker, 2a: Second marker (BHQ-2), 2b: Second marker (Cy5), 3: Target nucleic acid molecule (Target NA), 4: Nonspecific nucleic acid molecule, 5: Covalent bond

What is claimed is:

1. A method for quantifying target nucleic acid molecules in a nucleic acid-containing sample, comprising:
(a) preparing a sample solution comprising a nucleic acid-containing sample, a first nucleic acid molecule probe conjugated with a first marker, and a second nucleic acid molecule probe conjugated with a second marker;
(b) denaturing nucleic acid molecules in the sample solution prepared in (a);
(c) hybridizing the nucleic acid molecules in said sample solution, after (b);
(d) forming a covalent bond between two nucleic acid strands in the thus formed hybrid under a same condition, regarding the temperature and the salt concentration of the sample solution, as that of the hybrid formation in (c), after (c); and
(e) quantifying said target nucleic acid molecules by detecting a time course change in an optical characteristic of the first marker or the second marker in said sample solution, after (d);
wherein said first nucleic acid molecule probe comprises a nucleotide sequence complementary to said target nucleic acid molecule, said second nucleic acid molecule probe comprises a nucleotide sequence complementary to said first nucleic acid molecule probe, and at least either one of said first marker and said second marker is a substance whose optical characteristic is changed depending on whether or not said first nucleic acid molecule probe and said second nucleic acid molecule probe are hybridized.

2. The method for quantifying target nucleic acid molecules in a nucleic acid-containing sample target nucleic acid molecule quantification according to claim 1, wherein at least either one of said first marker and said second marker is a fluorescent dye, and said optical characteristic is detected through detection of a time course change in the fluorescence intensity emitting from said first marker or said second marker.

3. The method for quantifying target nucleic acid molecules in a nucleic acid-containing sample target nucleic acid molecule quantification according to claim 1, wherein the reaction for forming the covalent bond in (d) is a photochemical reaction.

4. The method for quantifying target nucleic acid molecules in a nucleic acid-containing sample target nucleic acid molecule quantification according to claim 3, wherein at least one nucleotide of said first nucleic acid molecule probe within the nucleotide sequence complementary to said target nucleic acid molecule is substituted by a photoreactive nucleotide derivative, and said covalent bond is formed via said photoreactive nucleotide derivative.

5. The method for quantifying target nucleic acid molecules in a nucleic acid-containing sample target nucleic acid molecule quantification according to claim 1, wherein at least one nucleotide of said first nucleic acid molecule probe in the nucleotide sequence complementary to said target nucleic acid molecule is substituted by 3-cyanovinylcarbazole nucleoside, and said covalent bond is formed by irradiation on said sample solution with light at 340 to 380 nm.

6. The method for quantifying target nucleic acid molecules in a nucleic acid-containing sample target nucleic acid molecule quantification according to claim 1, wherein a temperature of said sample solution at the time of covalent bond formation in (d) is within a range of Tm value±3° C.

7. The method for quantifying target nucleic acid molecules in a nucleic acid-containing sample target nucleic acid molecule quantification according to claim 2, wherein said target nucleic acid molecules are quantified through detection of a change in the fluorescence intensity of molecules existing in the focal area of a confocal optical system, and subsequent statistical analysis thereof to calculate the number of molecules of the first nucleic acid molecule probe or the second nucleic acid molecule probe hybridizing to said target nucleic acid molecules.

8. The method for quantifying target nucleic acid molecules in a nucleic acid-containing sample target nucleic acid molecule quantification according to claim 2, wherein said target nucleic acid molecules are quantified through detection of a fluctuation of the fluorescence intensity of molecules existing in the focal area in a confocal optical system, and subsequent statistical analysis thereof to calculate the number of molecules of the first nucleic acid molecule probe or the second nucleic acid molecule probe hybridizing to said target nucleic acid molecules.

9. The method for quantifying target nucleic acid molecules in a nucleic acid-containing sample target nucleic acid molecule quantification according to claim 8, wherein the fluctuation of the fluorescence intensity is analyzed by autocorrelation analysis.

10. The method for quantifying target nucleic acid molecules in a nucleic acid-containing sample target nucleic acid molecule quantification according to claim 1, wherein said first marker is a fluorescent substance and said second marker is a quenching substance for quenching fluorescence emitting from said fluorescent substance.

11. The method for quantifying target nucleic acid molecules in a nucleic acid-containing sample target nucleic acid molecule quantification according to claim 1, wherein said second marker is a fluorescent substance and said first marker is a quenching substance for quenching fluorescence emitting from said fluorescent substance.

12. The method for quantifying target nucleic acid molecules in a nucleic acid-containing sample target nucleic acid molecule quantification according to claim 1, wherein said first marker and said second marker are fluorescent dyes.

13. The method for quantifying target nucleic acid molecules in a nucleic acid-containing sample target nucleic acid molecule quantification according to claim 1, wherein said target nucleic acid molecule is a micro RNA.

14. The method for quantifying target nucleic acid molecules in a nucleic acid-containing sample target nucleic acid molecule quantification according to claim 1, wherein said target nucleic acid molecule is an siRNA.

15. A target nucleic acid molecule quantification kit comprising:
a first nucleic acid molecule probe comprising a nucleotide sequence complementary to the target nucleic acid molecule and conjugated with a first marker, and
a second nucleic acid molecule probe comprising a nucleotide sequence complementary to the first nucleic acid molecule probe and conjugated with a second marker;
wherein at least either one of said first marker and said second marker is a molecule whose optical characteristic is changed depending on whether or not said first nucleic acid molecule probe and said second nucleic acid molecule probe are hybridized, and at least one nucleotide of said first nucleic acid molecule probe within the nucleotide sequence complementary to said target nucleic acid molecule is substituted by a photoreactive nucleotide derivative.
